(19) United States
(12) Patent Application Publication
(10) Pub. No.: US 2024/0100253 A1
(43) Pub. Date: Mar. 28, 2024

(54) INCORPORATION OF ADDITIONAL SENSORS INTO ADAPTATION OF AID SYSTEMS

(71) Applicant: INSULET CORPORATION, Acton, MA (US)

(72) Inventors: Joon Bok LEE, Acton, MA (US); Yibin ZHENG, Hartland, WI (US); Jason O'CONNOR, Acton, MA (US); Ashutosh ZADE, San Diego, CA (US)

(21) Appl. No.: 18/466,211

(22) Filed: Sep. 13, 2023

Related U.S. Application Data
(60) Provisional application No. 63/375,561, filed on Sep. 14, 2022.

(51) Int. Cl. A61M 5/172; A61M 5/142

(52) U.S. Cl. CPC A61M 5/1723; A61M 5/14244; A61M 2202/0486; A61M 2230/04; A61M 2230/40

(57) ABSTRACT

Disclosed are techniques, devices and systems that provide adjustments to parameter settings of an insulin delivery algorithm based on inputs from a number of generic sensor devices. A majority of the generic sensor devices provide sensor readings that are unused as inputs to a drug deliver algorithm. The generic sensor devices may be operable to detect characteristics, such as changes in a state of a user. A processor may evaluate a sensor reading provided by a particular sensor with respect to a sensor baseline reading for the particular sensor. Using the result of the evaluation, the processor may calculate an adjustment to a parameter setting or settings of a medication delivery algorithm. A dosage of medication may be modified based on the adjustment of the parameter setting or settings.

### CROSS-REFERENCE TO RELATED APPLICATIONS

This applications claims priority to and the benefit of U.S. Application No. 63/375,561, filed Sep. 14, 2022, the entirety of which is incorporated herein.

### BACKGROUND

Adaptation of automated insulin delivery (AID) systems is critical to ensure the insulin therapies of each user are appropriately maintained as the user's insulin needs vary over time. Adaptation of AID systems based only on glucose and insulin delivery history may not provide sufficient granularity that enable control outcomes to accurately adapt AID systems.

It would be beneficial if there was a device or algorithm that took advantage of data available from multiple sensors that measure or generate data with sufficient granularity that enables an AID system to make accurate adaptations to the AID system.

### BRIEF SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

According to an example of the disclosed subject matter, a system is disclosed that includes a number of sensors, a memory, and a processor. Each sensor of the plurality of sensors may be configured to generate a respective output related to a state of a user. The memory may store a plurality of sensor baseline readings of respective sensors of the plurality of sensors. The processor, when executing the programming instructions, is operable to obtain a respective sensor reading output from a respective sensor of the number of sensors. The respective sensor reading and a respective sensor baseline reading that corresponds to the respective sensor in the stored plurality of sensor baseline readings may be evaluated. Based on the evaluation, an indication that an insulin delivery algorithm parameter is to be adjusted may be generated.

Another example of a non-transitory computer-readable medium is provided. A processor, when executing the programming instructions embodied in the non-transitory computer-readable medium, is operable to obtain a respective sensor reading output from a respective sensor of the number of sensors. The respective sensor reading and a respective sensor baseline reading that corresponds to the respective sensor in the stored plurality of sensor baseline readings may be evaluated. Based on the evaluation, an insulin delivery algorithm parameter may be adjusted, and an indication that an insulin delivery algorithm parameter is to be adjusted may be generated.

An example of a wearable drug delivery device is provided. The wearable drug delivery device may include a memory, communication circuitry, and a processor. The memory may store a number of sensor baseline readings of respective sensors of a number of sensors and a medication delivery algorithm. The communication circuitry may be operable to receive wireless signals. The processor may be coupled to the memory and the communication circuitry. The processor may be operable to execute programming instructions and when executing the programming instructions, is operable to obtain sensor readings from one or more of the plurality of sensors. Based on the received sensor readings and corresponding baseline sensor readings, the processor may determine that the received sensor readings from one or more of the plurality of sensors indicate that a parameter input to a medication delivery algorithm is to be modified. An adaptation parameter may be calculated based on a summation of an individual insulin delivery parameter generated for each respective one or more of the plurality of sensors. The parameter input to the medication delivery algorithm may be modified.

### BRIEF DESCRIPTION OF THE DRAWINGS

FIG. 1 illustrates an example of a drug delivery system that is suitable to implement the subject matter described herein.

FIG. 2 illustrates a non-drug delivery-related sensor network operable to communicate a drug delivery system.

FIG. 3 is a flowchart illustrating an example process that utilizes the outputs of respective non-drug delivery-related sensors according to the examples disclosed herein.

FIG. 4 shows a flowchart illustrating a detailed process for generating an adaptation parameter usable to modify parameters of a drug delivery algorithm.

### DETAILED DESCRIPTION

The following discussion provides a generalized framework to adapt parameters that affect the performance of a medication delivery algorithm (MDA) of an AID system based on readings provided by generic sensors other than those that provide inputs used in the determination of drug dosages by the MDA. The generic sensor readings are used to inform the parameters that control how the MDA responds to analyte sensor inputs, such as inputs from a continuous glucose monitor, a ketone monitor, or the like. The generic sensor readings when taken individually, in a combination of one or more, or in the aggregate, may indicate that the user has a higher or lower risk of either hypoglycemic or hyperglycemic glucose concentrations.

A type of medication delivery algorithm (MDA) may include an “artificial pancreas” algorithm-based system, or more generally, an artificial pancreas (AP) application that may be used in an AID system. For ease of discussion, the computer programs and computer applications that implement the medication delivery algorithms or applications may be referred to herein as an “AP application.” An AP application may be configured to provide automated delivery of insulin based on an analyte sensor input, such as signals received from an analyte sensor, such as a continuous blood glucose monitor (CGM), ketone sensor, or the like. The signals from the analyte sensor may contain blood glucose measurement values, timestamps, or the like.

The MDA may include a number of factors used in calculation of dosages of insulin to be provided by the automatic delivery of insulin. The different parameters may include a basal setting, a set point, a Q:R ratio of a cost function (e.g., a ratio of the cost of glucose deviations from a target BG level to the cost of insulin delivery deviations from an expected delivery amount), and a gain parameter, as well as others.

In addition, or alternatively, while the disclosed examples may be described with reference to a closed loop algorithmic implementation (i.e., no user involvement during routine operation), variations of the disclosed examples may be implemented to enable open loop use (i.e., with user involvement) or a hybrid closed loop use (i.e., very limited user involvement). The open loop implementations allow for use of different modalities of delivery of insulin such as smart pen, syringe, or the like. For example, the disclosed AP application and algorithms may be operable to perform various functions related to open loop operations, such as the generation of prompts requesting the input of information such as diabetes type, weight, or age. Similarly, a dosage amount of insulin may be received by the AP application or algorithm from a user via a user interface. Other open-loop actions may also be implemented by adjusting user settings, or the like in an AP application or algorithm.

Systems, devices, computer readable media and methods in accordance with the present disclosure are now described more fully hereinafter with reference to the accompanying drawings, where one or more examples are shown. The systems, devices, and methods described herein may be embodied in many different forms and are not to be construed as being limited to the examples set forth herein. Instead, these examples are provided so the disclosure is thorough and complete, and may fully convey the scope of the techniques and devices to those skilled in the art. Each of the systems, devices, media, and methods disclosed herein provides one or more advantages over conventional systems, components, and methods.

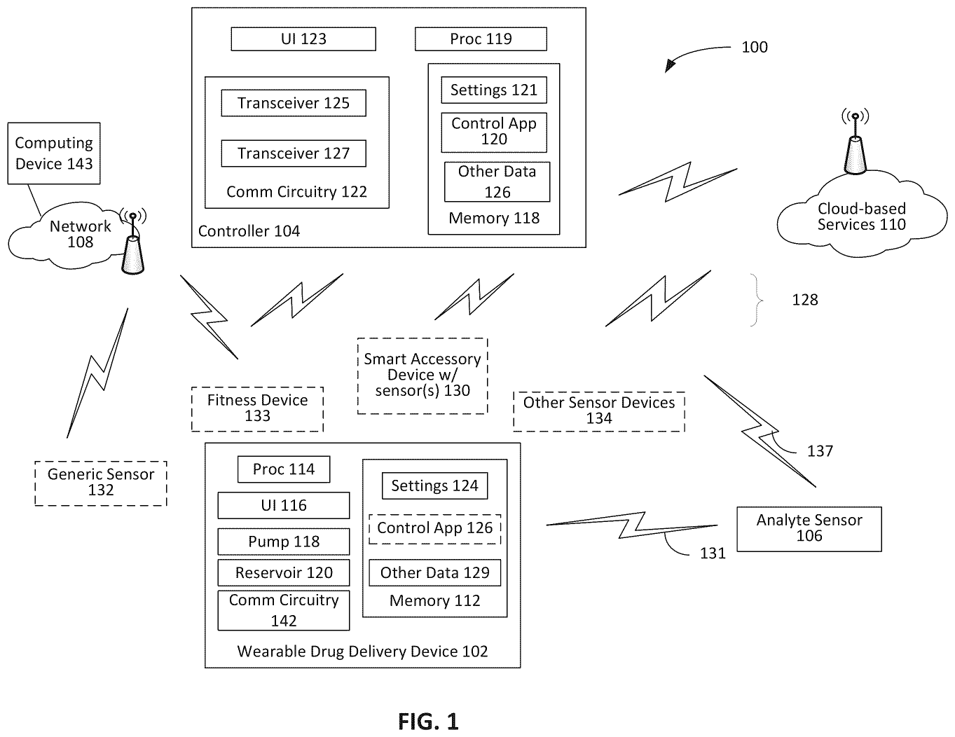

FIG. 1 illustrates a drug delivery system.

In some examples, the drug delivery system 100 is suitable for delivering insulin to a user in accordance with the disclosed embodiments. The drug delivery system 100 may include a wearable drug delivery device 102, a controller 104, and an analyte sensor 106.

The wearable drug delivery device 102 may be a wearable device that is worn on the body of the user. The wearable drug delivery device 102 may be a multi-part device. For example, the wearable drug delivery device 102 may have a first part and a second part that couple together. The first part and/or second part may fit into or slide into a tray or cradle that is adhered to the user's body, and the first part and/or second part may be removable from the tray. If using a first part and a second part, the first part may comprise reusable components (e.g., electronic circuitry, processor, memory, a pump mechanism, and potentially a rechargeable battery), and the second part may comprise disposable components (e.g., a reservoir, a needle and/or cannula, a disposable battery, and other portions or components that come into contact with the liquid drug or medicament). Moreover, the first part and the second part may contain their own housing or may combine together to form a single housing. The wearable drug delivery device 102 may be directly coupled to a user (e.g., directly attached to a body part and/or skin of the user via an adhesive, directly, via the tray, or the like). In an example, a surface of the wearable drug delivery device 102 or a tray into which the wearable drug delivery device 102 couples may include an adhesive to facilitate attachment to the skin of a user.

The wearable drug delivery device 102 may include a processor 114. The processor 114 may be implemented in hardware, software, or any combination thereof. The processor 114 may, for example, be a microprocessor, a logic circuit, a field programmable gate array (FPGA), an application specific integrated circuit (ASIC), or a microprocessor coupled to a memory. The processor 114 may maintain a date and time as well as be operable to perform other functions (e.g., calculations, or the like). The processor 114 may be operable to execute a control application 126 stored in the memory 112 that enables the processor 114 to direct operation of the wearable drug delivery device 102. The control application 126 may control insulin delivery to the user per an MDA control approach as described herein. For example, the control application 126 may be an MDA algorithm. The memory 112 may hold settings 124 for a user, such as specific factor settings, subjective insulin need parameter settings, MDA settings, such as maximum insulin delivery, insulin sensitivity settings, total daily insulin (TDI) settings, insulin decay settings, and the like. The memory may also store other data 129, such as total daily insulin values, blood glucose measurement values from analyte sensor 106 or controller 104, insulin dosage amounts (both basal and bolus) and the like from previous minutes, hours, days, weeks, or months. The analyte sensor 106 may be operable to collect a user's physiological condition data, such as the blood glucose measurement values and a timestamp, that may be shared with the wearable drug delivery device 102, the controller 104 or both. For example, the communication circuitry 142 of the wearable drug delivery device 102 may be operable to communicate with the analyte sensor 106 and the controller 104 as well as the devices 130, 133 and 134. The communication circuitry 142 may be operable to communicate via Bluetooth, cellular communication, and/or other wireless protocols. While not shown, the memory 112 may include both primary memory and secondary memory. The memory 112 may include random access memory (RAM), read only memory (ROM), optical storage, magnetic storage, removable storage media, solid state storage, or the like.

The wearable drug delivery device 102 may include a reservoir 120. The reservoir 120 may be operable to store drugs, medications or therapeutic agents suitable for automated delivery. A fluid path to the user may be provided via tubing and a needle/cannula (not shown). The fluid path may, for example, include tubing coupling the wearable drug delivery device 102 to the user (e.g., via tubing coupling a needle or cannula to the reservoir 120). The wearable drug delivery device 102 may be operable based on control signals from the processor 114 to expel the drugs, medications or therapeutic agents, such as insulin, from the reservoir 120 to deliver doses of the drugs, medications or therapeutic agents, such as the insulin, to the user via the fluid path. The processor 114 may be operable to cause insulin to be expelled from the reservoir 120.

There may be one or more communications links 128 with one or more devices physically separated from the wearable drug delivery device 102 including, for example, a controller 104 of the user and/or a caregiver of the user and/or a sensor 106. The communication links 128 may include any wired or wireless communication link operating according to any known communications protocol or standard, such as Bluetooth®, Wi-Fi, a near-field communication standard, a cellular standard, or any other wireless protocol. The analyte sensor 106 may communicate with the wearable drug delivery device 102 via a wireless communication link 131 and/or may communicate with the controller 104 via a wireless communication link 137.

The wearable drug delivery device 102 may also include a user interface 116, such as an integrated display device for displaying information to the user, and in some embodiments, receiving information from the user. For example, the user interface 116 may include a touchscreen and/or one or more input devices, such as buttons, knobs, or a keyboard that enable a user to provide an input.

In addition, the processor 114 may be operable to receive data or information from the analyte sensor 106 as well as other devices that may be operable to communicate with the wearable drug delivery device 102.

The wearable drug delivery device 102 may interface with a network 108. The network 108 may include a local area network (LAN), a wide area network (WAN) or a combination therein. A computing device 143 may be interfaced with the network, and the computing device may communicate with the insulin delivery device 102. The computing device 143 may be a healthcare provider device through which a user's controller 104 may interact to obtain information, store settings and the like. The AID algorithm operating, as or in cooperation with, the control application 120 may present a graphical user interface on the computing device 143 enabling the input and presentation of information related to the AID algorithm.

The drug delivery system 100 may include an analyte sensor 106 for sensing the levels of one or more analytes of a user. The analyte levels may be used as physiological condition data and be sent to the controller 104 and/or the wearable drug delivery device 102. The sensor 106 may be coupled to the user by, for example, adhesive, or the like and may provide information or data on one or more medical conditions and/or physical attributes of the user. The sensor 106 may be a continuous glucose monitor (CGM), or another type of device or sensor that provides blood glucose measurements that is operable to provide blood glucose concentration measurements. The sensor 106 may be physically separate from the wearable drug delivery device 102 or may be an integrated component thereof. The sensor 106 may provide the processor 114 and/or processor 119 with physiological condition data indicative of measured or detected blood glucose levels of the user. The information or data provided by the sensor 106 may be used to modify an insulin delivery schedule and thereby cause the adjustment of drug delivery operations of the wearable drug delivery device 102.

The drug delivery system 100 may also include the controller 104. In the depicted example, the controller 104 may include a processor 119 and a memory 118. The controller 104 may be a special purpose device, such as a dedicated personal diabetes manager (PDM) device. The controller 104 may be a programmed general-purpose device that is a portable electronic device, such as any portable electronic device, smartphone, smartwatch, fitness device, tablet, or the like including, for example, a dedicated processor, such as micro-processor, or the like. The controller 104 may be used to program or adjust operation of the wearable drug delivery device 102 and/or the sensor 106. The processor 119 may execute processes to manage a user's blood glucose levels and for controlling the delivery of the drug or therapeutic agent to the user. The processor 119 may also be operable to execute programming code stored in the memory 118. For example, the memory 118 may be operable to store a control application 120, such as an AID algorithm for execution by the processor 119. The control application 120 may be responsible for controlling the wearable drug delivery device 102, including the automated delivery of insulin based on recommendations and instructions from the AID algorithm, such as those recommendations and instructions described herein.

The memory 118 may store one or more applications, such as control application 120, and settings 121 for the insulin delivery device 102 like those described above. In addition, the memory 118 may be operable to store other data and/or computer programs 126, such as drug delivery history, blood glucose measurement values over a period of time, total daily insulin values, and the like. For example, the memory 118 is coupled to the processor 119 and operable to store programming instructions, such as the control application 120 and settings 121, and data, such as other data 126, related to blood glucose measurements of a user and/or data related to an amount of insulin expelled by the wearable drug delivery device 102.

The controller 104 may include a user interface (UI) 123 for communicating with the user. The user interface 123 may include a display, such as a touchscreen, for displaying information. The touchscreen may also be used to receive input when it is a touch screen. The user interface 123 may also include input elements, such as a keyboard, buttons, knobs, or the like. In an operational example, the user interface 123 may include a touchscreen display controllable by the processor 119 and be operable to present the graphical user interface, and in response to the received input, the touchscreen display is operable to generate a signal indicative of the subjective insulin need parameter. The touchscreen display, under control of the processor 119, may be operable to receive inputs to computer applications, such as a consumer food tracker, carbohydrate calculator, mapping applications based on a GPS, Wi-Fi or Bluetooth location of a user, or the like.

The controller 104 may interface via a wireless communication link of the wireless communication links 128 with a network, such as a LAN or WAN or combination of such networks that provides one or more servers or cloud-based services 110 via communication circuitry 122. The communication circuitry 122, which may include transceivers 127 and 125, may be coupled to the processor 119. The communication circuitry 122 may be operable to transmit communication signals (e.g., command and control signals) to, and receive communication signals (e.g., via transceivers 127 or 125) from, the wearable drug delivery device 102 and the analyte sensor 106. In an example, the communication circuitry 122 may include a first transceiver, such as 125, that may be a Bluetooth transceiver, which is operable to communicate with the communication circuitry 122 of the wearable drug delivery device 102, and a second transceiver, such as 127, that may be a cellular or Wi-Fi transceiver operable to communicate via the network 108 with computing device 143 or with cloud-based services 110.

The cloud-based services 110 may be operable to store user history information, such as blood glucose measurement values over a set period of time (e.g., days, months, years), a drug delivery history that includes insulin delivery amounts (both basal and bolus dosages) and insulin delivery times, types of insulin delivered, indicated or detected meal times, blood glucose measurement value trends or excursions or other user-related diabetes treatment information, specific factor settings including default settings, present settings and past settings, or the like.

Other devices, like smart accessory device 130 (e.g., a smartwatch or the like), fitness device 133 and other sensor devices 134 may be part of the drug delivery system 100. For example, the other sensor devices 134 may be at least one of a heart rate monitor, an oxygen sensor, a carbohydrate calculator, a pedometer, a meal tracker application, a blood glucose sensor, a ketone sensor, or the like. These sensor devices 130, 133 and 134 may communicate with the wearable drug delivery device 102 to provide sensor readings and other values related to readings made by the respective sensor devices to the wearable drug delivery device 102. These sensor devices 130, 133 and 134 may execute computer programming instructions to perform some of the control functions otherwise performed by processor 114 or processor 119. These sensor devices 130, 133 and 134 may include user interfaces, such as touchscreen displays for displaying information such as accelerometer data, number of steps, heart rate, such as those described with reference to the examples of FIGS. 1-3. The display may, for example, be operable to present a graphical user interface for receiving input, such as receiving input of a meal size, an estimate of the number of carbohydrates of a meal, or the like. These sensor devices 130, 133 and 134 may also have wireless communication connections with the sensor 106 to directly receive blood glucose level data or receive in parallel a presentation of the graphical user interface as shown in FIG. 1. Furthermore, the smart accessory devices 130 may be operable to execute one or more programming applications that present information about when a user eats, an amount of sleep the user gets, an amount of screentime the of the user, as well as applications related to stress levels, number of times the user checked their blood glucose level, meal size, meal fat content, meal frequency, weekly restaurant visits, number of days of illness, days of stress, number of days of participating in meditation and calming exercises, medications and supplements used by the user, or the like. Examples of the other devices 134 may include a smart scale (such as Renpho® Bluetooth scale or the like), exercise monitors/timers, heart rate and/or heart rate variability monitors, blood oxygen monitors, and the like.

In an operational example, a processor, such as 114 or 119, in the drug delivery system may be operable to receive, via communication circuitry (such as 142 of the wearable drug delivery device 102 or 122 of the controller 104) physiological data inputs from devices external to the drug delivery system, such as 133, 130 and other devices 134. The processor may utilize the physiological data inputs to validate the evaluation of the glucose measurement values over a period of time as discussed in more detail with reference to the following examples.

In another operational example, the controller 104 may be operable to execute programming code that causes the processor 119 of the controller 104 to also perform different functions. For example, the processor 119 of the controller 104 may execute an AID algorithm that is one of the control applications 120 stored in the memory 112 or memory 118. The processor may be operable to present, on a user interface that is at least one component of the user interface 123. The user interface 123 may be a touchscreen display controlled by the processor 119, and the user interface 123 is operable to present a graphical user interface that offers an input of a subjective insulin need parameter usable by the AID algorithm. The processor 119 may cause presentation of a graphical user interface offering an input device that enables input of a subjective insulin need parameter. The AID algorithm may generate instructions for the pump 118 to deliver basal insulin to the user, or the like.

The processor 119 is also operable to collect physiological condition data related to the user from sensors, such as the analyte sensor 106 or heart rate data, for example, from the fitness device 133 or the smart accessory device 130. In an example, the processor 119 executing the AID algorithm may determine a dosage of insulin to be delivered based on the collected physiological condition of the user and a specific factor determined based on the subjective insulin need parameter. The processor 119 may output a control signal via one of the transceivers 125 or 127 to the wearable drug delivery device 102. The outputted signal may cause the processor 114 to deliver command signals to the pump 118 to deliver an amount of medicament related to the determined dosage of insulin in the reservoir 120 to the user based on an output of the AID algorithm.

The drug delivery system 100 functions according to an operational cycle that repeats after a duration of time or upon completion of a previous cycle. As mentioned, each operational cycle, the analyte sensor 106 of the drug delivery system 100 may obtain one or more measurements of an analyte of a user's blood, such as a blood glucose value and/or a ketone level value. Still during the operational cycle, the analyte measurement value(s) may be transmitted by communication circuitry (not shown) in the analyte sensor 106 to a processor (e.g., processor 119 at the controller 104 or the processor 114 of the wearable drug delivery device 102, or both). The processor 119 or 114 may evaluate the received analyte measurement value, and according to an algorithm executed by the processor 119 or 114, a dosage of a liquid medication, such as those listed herein, may be recommended for delivery to the user. The wearable drug delivery system 100 is operable to deliver the recommended dosage to the user. Upon completion of the delivery of the recommended dosage, the current operational cycle ends and a new operational cycle begins in which the above processes are repeated. An operational cycle may have a duration of approximately 5 minutes, but other durations are available, such as 2 minutes, 10 minutes, or the like. In some examples, the analyte sensor 106 may provide multiple analyte measurement values during an operational cycle and the processor 119 or 114 may or may not process each received analyte measurement value.

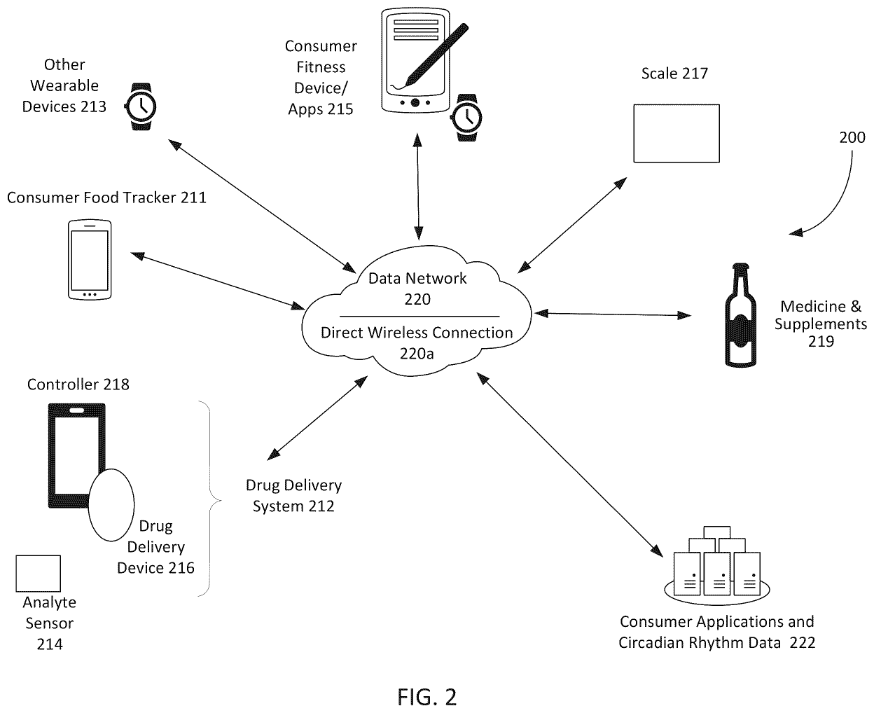

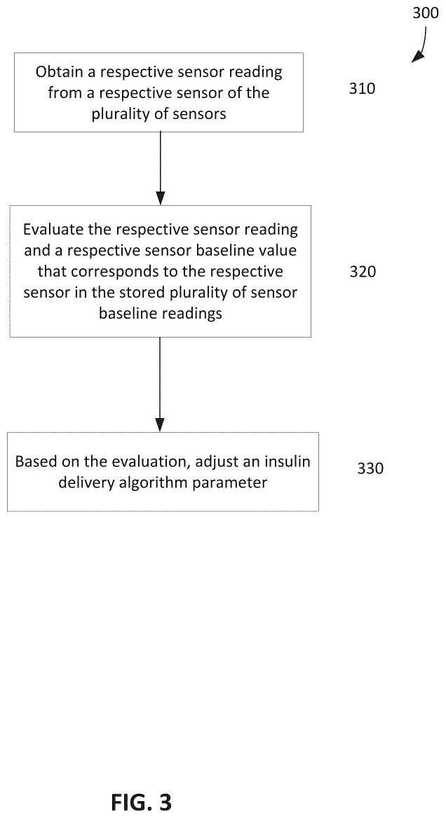

A wearable drug delivery device 102 typically has a lifecycle that is based on the amount of liquid drug that is stored in a reservoir 120 of the wearable drug delivery device 102 and/or the amount of the liquid drug delivered to the user. An AID application or algorithm may use a number of parameters, such as blood glucose measurement values, total daily insulin, insulin onboard, and the like, when making the determination of an amount of the liquid drug to have delivered. In an operational example, the processor 119 of the controller 104 may be operable to evaluate the effectiveness of the AID algorithm's control of the drug delivery device 102. For example, the processor 119 may be operable to utilize an indication of carbohydrates to determine customized glycemic index values for different food items or combinations of food items (e.g., meals, such as breakfast, lunch or dinner, as well as snacks or sandwiches and the like) and using programming code may execute the functions or calculations described in more detail with reference to the examples of FIGS. 2 and 3.

While the system 100 is described with reference to delivery of insulin and the use of an AID algorithm, the system 100 may be operable to implement a drug delivery regimen via a medication delivery algorithm using a number of different liquid or therapeutic drugs. A liquid drug may be or include any drug in liquid form capable of being administered by a drug delivery device via a subcutaneous cannula, including, for example, insulin, glucagon-like peptide-1 (GLP-1), pramlintide, glucagon, co-formulations of two or more of GLP-1, pramlintide, and insulin; as well as pain relief drugs, such as opioids or narcotics (e.g., morphine, or the like), methadone, arthritis drugs, hormones, such as estrogen and testosterone, blood pressure medicines, chemotherapy drugs, fertility drugs, or the like.

FIG. 2 illustrates an example of an ecosystem of sensors that produce sensor reading or tracking information that produces data values (typically) unused (e.g. sensor readings or tracking information other than blood glucose measurements) by an AID system and MDA according to the examples disclosed herein. In some embodiments, the majority of sensors in the system are (typically) unused by the AID system or MDA, in particular wherein majority relates to more than half the sensors. For example, the sensor ecosystem 200 may include a number of generic sensors that provide a wide range of sensor readings. The generic sensors may include, for example, an accelerometer, a gyroscope, a pedometer, an activity monitor, a blood oxygen (02) sensor, heart rate monitor, a sleep monitor, a stress monitor, a caffeine intake tracker, an alcohol intake tracker, a water intake tracker, a meal frequency counter, a meal content calculator, a carbohydrate calculator, a clock, an ambient light detector, a screen time tracker, an environmental temperature monitor, a moisture sensor, a sweat sensor, or the like.

For example, the sensor ecosystem 200 may include a consumer food tracker 211, a drug delivery system 212, a consumer fitness devices and applications 215, a scale 217, medicine and supplements 219, a consumer applications and circadian rhythm data 222, or the like. The drug delivery system 212 may include an analyte sensor, a drug delivery device 216 and a controller 218 that are similar to those discussed with reference to FIG. 1.

One or more components of the drug delivery system 212 include communication circuitry (not shown in this example) that is operable to communicate with the data network 220 and/or directly via a direct wireless connection 220a (e.g., Bluetooth, Wi-Fi, or the like) with one or more of devices in the sensor ecosystem 200. For example, the consumer food tracker 211 and the consumer fitness devices and applications 215, and other wearable devices 213 may provide data (as shown in Table 2 below) such as accelerometer sensor readings, GPS information that allows for sensor readings related to restaurant colocation frequency or weekly restaurant visit frequency (number of times in the previous week/7-days), meal frequency, meal size, meal carbohydrate content, meal fat content, meal protein content, caffeine intake, number of hours of sleep, or the like. These kinds of data are typically not included as an input for setting of parameter values as used in a medication delivery algorithm (or AID system). The data provided may be used to adapt parameter settings such as a glucose control target, clinical input parameters (e.g., TDI), cost function parameters (such as an R(t) value of a Q:R cost function ratio), an insulin-to-carbohydrate ratio or a correction factor (e.g., insulin sensitivity). Other factors, such as basal dosage (e.g., B(t)) and system gain (e.g., K(t)) may also be adapted using the techniques described herein.

Returning to FIG. 2, the scale 217 and the analyte sensor 214 may measure physiological attributes of the user or may receive from the user as inputs to the respective device's information relevant to the user's physiological attributes.

In the process described with reference to FIG. 3 and beyond, a generic sensor n may provide readings Sₙ (i.e., generic sensor n value) over time that deviate from a baseline sensor reading for the specific sensor and may also provide an indication that the user is currently or may experience a specific hypoglycemic or hyperglycemic incidence. The generated indication may be based on the specific sensor deviation alone or in combination with other sensor deviations from baseline.

The processor may receive sensor readings as inputs from a number of different sensors and based on an evaluation of the received sensor readings, the processor may tune the AID system by applying various values for the various parameters of the AID system/MDA based on output(s) of generic sensor(s) Sₙ. For example, the processor may make changes to (e.g., increasing or decreasing) one or more of the following tuning parameter settings of the AID system/MDA; glucose control target; blood glucose setpoint SP(t); input clinical parameters (such as total daily insulin (TDI)); the Q:R ratio of a cost function, for example by adjusting the value R(t) of a cost function; insulin-to-carbohydrate (IC) ratio; correction factor (also referred to as insulin sensitivity), basal dosage rate B(t); gain K(t), or the like. With regard to the Q:R ratio and R(t) of a cost function, the R value is a function of time and represents a coefficient to an insulin cost determined based on (potential) deviations of insulin delivery amounts from anticipated insulin delivery amounts.

FIG. 3 illustrates a process that determines whether an insulin delivery algorithm parameter is to be adjusted. The process 300 may be executed by a processor within a drug delivery system. For example, the processor and memory as part of a wearable drug delivery device may be operable to receive inputs directly or indirectly (e.g., via controller 104) from a number of sensors configured to detect a physiological condition (also referred to as a state of a user) of a user. The memory may be operable to store a number of sensor baseline readings of respective sensors of the number of sensors.

For example, the processor when executing programming instructions that implement the process 300 of FIG. 3 may be operable to receive a respective sensor reading from a respective sensor Sₙ of the plurality of sensors S (Step 310). The processor may be operable to receive a respective sensor reading continuously, at a periodic interval based on a request from the processor, or according to a scheduled delivery. For example, some of the sensors may run continuously and provide substantially real-time values, such as a heart rate monitor, while others may generate an output (related to the state of a user) every wearable drug delivery operational cycle (e.g., every 5 minutes), such as a blood glucose monitor. Alternatively, the sensor may have threshold settings and may only transmit an output when the threshold is reached or crossed, such as a stress monitor, or the like.

The respective sensor reading Sₙ(t) and a respective sensor baseline reading S_{n,baseline} that corresponds to the respective sensor in the stored plurality of sensor baseline readings may be evaluated (Step 320). For example, as shown in Equation 1 below, the processor may execute a function in which the processor determines a sensor reading difference between the respective sensor reading and the respective sensor baseline reading (the result being a first difference value) and determine a maximum sensor difference utilizing the respective sensor baseline reading and a maximum sensor output value (S_{n,max})(the result being a second difference value). The maximum sensor output value (S_{n,max}) may be maximum output the sensor is expected to output and the sensor baseline reading S_{n,baseline} may be the average or median reading (or output value) of the sensor. The average or median reading may be determined over a time frame. The time frame may be a number of hours, days, weeks, months or years. The time frame may be a rolling-window or may be determined or a specific time frame, e.g. a time frame for which settings for AID (such as a Q:R ratio) were determined. The processor may determine a quotient of the first difference value divided by the second difference value. The quotient may be multiplied by a weighting factor Wₙ that is specific to the respective sensor Sₙ. For example, a heart rate output of a Fitbit® may have a particular weighting, while a heart rate/blood oxygen combination monitor may have a different weighting for the heart rate monitor result value and yet a different weighting for the blood oxygen monitor result value. The weighting factor Wₙ may be determined from clinical trials or demographic assessments, an evaluation of user history for the respective sensor to isolate strong predictors for hypoglycemia or hyperglycemia. In some embodiments, the weighting factors are determined by regression analysis or machine learning. The result of the multiplication of the weighting factor Wₙ and the quotient may be a parameter adjustment value P_{Sn} that is attributable the respective sensor (Sₙ).

An example equation that may be implemented in this process is illustrated as follows:

$${P}_{S n} ( t ) = {W}_{n} \frac{{s}_{n} ( t ) - {s}_{n , baseline}}{{s}_{n , max} - {s}_{n , baseline}} \qquad ( Equation 1 )$$

where the values P_{Sn}(t) is the parameter adjustment value for the respective sensor (Sₙ) at time t, Wₙ is the weighting for the respective sensor (Sₙ), and the numerator is a difference of the sensor reading (Sₙ(t)) at time t between the respective sensor reading and the respective sensor baseline reading (S_{n,baseline}), and the denominator is a maximum sensor difference between a maximum sensor output value (S_{n,max}) and the respective sensor baseline reading (S_{n,baseline}). The numerator is the first difference value referenced above and the denominator is the second difference value referenced above. Note that the units cancel in P_{Sn} of Equation 1.

The output received from each generic sensor can thus be utilized based on standard and upper bound limits, and the respective sensor's expected impact on a user's hyperglycemia or hypoglycemia thresholds.

For example, at 330, based on the evaluation, the processor may adjust an insulin delivery algorithm parameter.

The expected impact on hyperglycemia or hypoglycemia can then be executed as an adaptation parameter that is thus multiplied to each tuning parameter in an AID system listed above, similar to the following:

$$A ( t ) = \frac{{\sum }_{i = 1}^{n} {P}_{S n} ( t )}{n} \qquad ( Equation 2 )$$

where the adaptation parameter A(t) is an average of the available impact factors for n number of sensors. The adaptation parameter A(t) may be applied to each, or one or more, of the tuning parameter settings mentioned above. Accordingly, in some embodiments, the adaptation parameter is determined by determining a parameter adjustment value individually for a plurality of sensors, in particular each sensor of the plurality of sensors, and determining the adaptation parameter as the average of the parameter adjustment values.

In one particular example, two generic sensors can be implemented into the AID system—an accelerometer and a sensor describing a frequency of the user in a GPS location close to restaurants (e.g., a computer application that evaluates GPS locations with locations frequented by a user). Such inputs from an accelerometer, a sensor that evaluates GPS signals, or any other generic sensor, can be utilized by a medication delivery algorithm by applying a weighting factor to the output of each sensor based on their association with the user state that the drug delivery is designed to control. This weighting methodology provides a robust methodology for any generic sensor to be incorporated into the drug delivery algorithm without the need for detailed model identifications. In the described examples, since each sensor gets a weighted factor, the result of A(t) is a weighted sum of each weighted factor P_{Sn}(t).

Using the above framework, Table 1 below shows an example of different readings for the respective sensors and the sensor's respective sensor readings. The weighting Wₙ can, for instance, result in the following establishment of each expected impact on hyperglycemia or hypoglycemia:

TABLE 1

| Sensor | Weight Wₙ | S_{n, max} | S_{n, baseline} | Sₙ(t) |
|---|---|---|---|---|
| P₁) | −0.01 | 4 miles/hour | 0.1 mph | 2 mph |
| Accelerometer | (i.e., current | (mph) | (The user | (e.g., user is |
| (n = 1) | “Max” expected | (At an exercise | typically moves | currently |
| | reading may | speed of 4 mph | on average 0.1 | exercising at 2 |
| | result in ~1% | or more, the user | mph) | mph) |
| | impact in | experiences ~1% | | |
| | overall glucose) | reduction in the | | |
| | | user's overall | | |
| | | glucose) | | |
| P₂) Restaurant | +0.0005 | 3 | 1 | 2 |
| colocation | (when user is at | (the user may | (the user | (this is the |
| frequency | this restaurant, | visit this | typically visits | second time the |
| (n = 2) | there is a ~0.05% | restaurant at | this restaurant | user is |
| | impact for | most 3 times a | once a week | approaching this |
| | increased | week) | without any | restaurant this |
| | glucose of the | | impact on | week) |
| | user) | | glucose) | |

In this example, based on clinical data or evaluation of the user's history with respect to the sensor readings output by the accelerometer or the restaurant colocation frequency, the respective correlation of the sensor's reading to a user's blood glucose concentration (also, referred to as glucose concentration) may be determined. For example, the accelerometer may have a negative correlation with glucose concentration—specifically, it is typically expected that the higher readings in the accelerometer result in higher likelihood of hypoglycemia. Alternatively, the restaurant colocation frequency may have a positive correlation with glucose concentration—specifically, it is expected that a higher frequency of presence in restaurants (i.e., colocation) results in a higher likelihood of hyperglycemia.

Then, the impact on hyperglycemia or hypoglycemia by Pₙ can be defined as follows:

$${P}_{1} = - 0.01 \frac{2 - 0.1}{4 - 0.1} = - 0.0 \; 487 - accelerometer \\ {P}_{2} = 0.0005 \frac{2 - 1}{3 - 1} = 0.0 \; 0025 - restaurant colocation frequency$$

Then, the adjustment factor A(t) as shown in Equation 2 above can be calculated as an average of available impact factors:

$$A ( t ) = \frac{- 0.0487 + 0.00025}{2} = - 0.0231 .$$

The −0.0231 value can then be translated into an adjustment of the user's various tuning parameters (also referred to as insulin delivery algorithm parameter value) within an AID system as a bias towards lower insulin delivery by 2.3% for the current cycle (i.e., 2.3% increase in glucose target, 2.3% decrease in input clinical parameters, 2.3% increase in Q:R ratio, 2.3% increase in IC ratio, 2.3% increase in correction factor, and the like). Accordingly, in some embodiments, adjusting the insulin delivery algorithm parameter value includes appraising (i.e. selecting) a value of the insulin delivery algorithm parameter value that is to be adjusted; and in response to the appraisal that the insulin delivery algorithm parameter is to be adjusted, produce a signal identifying the insulin delivery algorithm parameter value to be adjusted. Furthermore, in some embodiments, adjusting the insulin delivery algorithm parameter value further includes evaluating, in particular in response to the signal, other respective sensor readings received from the other respective sensors of the plurality of sensors. Further, based on the other respective sensor readings a respective insulin delivery parameter value for each of the other respective sensors of the plurality of sensors may be established. Additionally, using the insulin delivery parameter value and each respective insulin delivery parameter value of all of the other respective sensors of the plurality of sensors, an adaptation parameter usable by a medication delivery algorithm may be generated, in particular wherein the adaptation parameter is usable by the medication delivery algorithm to recalculate a parameter setting of the medication delivery algorithm. In some embodiments, adjusting the insulin delivery algorithm parameter value includes appraising a value of the insulin delivery algorithm parameter value that is to be adjusted and in response to the appraisal that the insulin delivery algorithm parameter is to be adjusted, adjust the insulin delivery algorithm parameter, in particular wherein adjusting the insulin delivery algorithm parameter includes producing a signal identifying the insulin delivery algorithm parameter value to be adjusted.

The different sensors may have different impacts and be assigned different sensor impacts that may be adjusted over time as the processor receives further outputs from the respective sensors. For example, as represented in Table 2, the processor may use further sensor outputs from sensors, such as those named in Table 2, to calibrate the sensor baseline reading (S_{n,baseline}).

TABLE 2

| | | | Sn, | | |
|---|---|---|---|---|---|
| | | | baseline | | |
| | | Sn, Max | (sensor | | |
| | Sensor impact | (typical | reading | Sn(t) | |
| | on insulin | “Max” | with no | (current | |
| | need at Sn, | reading for | glucose | sensor | Pn (Impact on Hyper |
| Sensor Name | Max reading | sensor) | impact) | reading) | or Hypoglycemia) |
| Accelerometer | −0.01 | 4 | 1 | 3 | −0.006666667 |
| (m/s) | | | | | |
| Weekly | 0.005 | 3 | 1 | 3 | 0.005 |
| Restaurant Visit | | | | | |
| Frequency | | | | | |
| (#/last week) | | | | | |
| meal frequency | 0.05 | 6 | 3 | 4 | 0.016666667 |
| (#/last 24 hours) | | | | | |
| meal size (g | 0.2 | 350 | 200 | 260 | 0.08 |
| CHO/last 24 | | | | | |
| hours) | | | | | |
| meal fat content | 0.1 | 100 | 40 | 80 | 0.066666667 |
| (g/last 24 hours) | | | | | |
| number of times | 0.025 | 3 | 1 | 4 | 0.0375 |
| caffeine is | | | | | |
| ingested (#/last | | | | | |
| 24 hours) | | | | | |
| Hours of Sleep | −0.025 | 10 | 8 | 6 | 0.025 |
| (h/last 24 hours) | | | | | |
| Number of BG | −0.02 | 6 | 2 | 3 | −0.005 |
| measurements | | | | | |
| (#/last 24 hours) | | | | | |
| Illness (# days | 0.05 | 3 | 1 | 1 | 0 |
| ill/last week) | | | | | |
| Stress (# days | 0.05 | 3 | 1 | 1 | 0 |
| stressed/last | | | | | |
| week) | | | | | |
| | | | | Final impact | 0.021916667 |

| | target | input TDI | Q:R ratio | IC ratio | Correction Factor |
|---|---|---|---|---|---|
| original | 110 | 90 | 9000 | 9 | 20 |
| modified | 107.5891667 | 91.9725 | 8802.75 | 8.80275 | 19.56166667 |

Accordingly, in some embodiments, the output generated by the sensor(s) related to a state of a user includes at least one of a user's stress levels, a user's speed of movement, a number of times the user checked their blood glucose level, a number of glucose measurements, a number of caffeine ingestions, a meal size, a meal fat content, a meal frequency, a number (weekly) restaurant visits, a number of days of illness, a number of days of stress, a number of days of participating in meditation and calming exercises, a medications and supplements used by the user, and/or a hours of sleep.

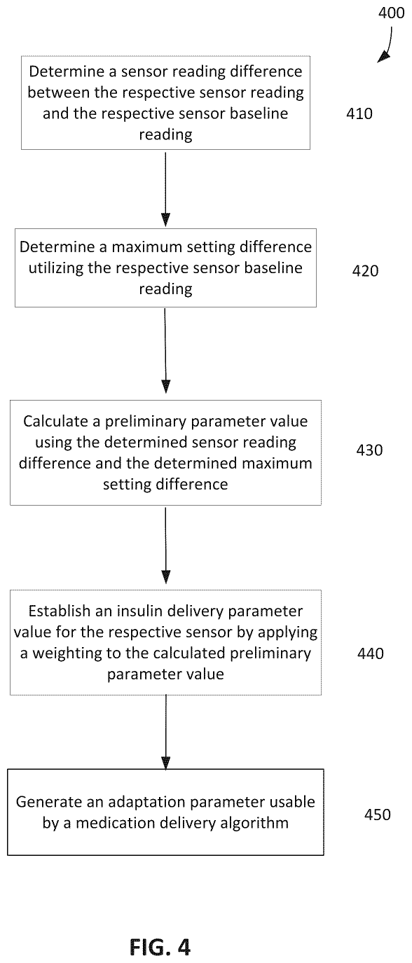

As can be seen in Table 2 above, the output from each respective sensor may be different and have different units. FIG. 4 illustrates a flowchart that implements a process for evaluating the respective sensor reading and the respective sensor baseline reading that corresponds to the respective sensor in the stored plurality of sensor baseline readings according to examples discussed herein. The process 400 tracks additional exemplary steps for implementing Equation 1, reproduced below for convenience. In an example, Equation 1 may be used to characterize a parameter value (Pₙ) attributable to the sensor reading (Sₙ) at time t as shown in the following:

$${P}_{S n} ( t ) = {W}_{n} \frac{{s}_{n} ( t ) - {s}_{n , baseline}}{{s}_{n , max} - {s}_{n , baseline}} \qquad ( Equation 1 )$$

where Sₙ(t) is a current sensor reading from sensor Sₙ, S_{n,baseline} is the user's average or typical value of the sensor output, and S_{n,max} is the maximum change of the sensor reading, as explained above. In step 410, the numerator of Equation 1, a sensor reading difference between the respective sensor reading and the respective sensor baseline reading is determined, while, at step 420, in the denominator, a maximum setting difference utilizing the respective sensor baseline reading is determined. The parameter value (Pₙ) does not have any units as the units cancel out in Equation 1. At 430, for example, the processor may calculate the quotient of the division as a preliminary parameter value using the determined sensor reading difference and the determined maximum setting difference. The insulin delivery parameter value P_{Sn}(t) is established for the respective sensor by applying a weighting to the calculated preliminary parameter value.

The parameter value P_{Sn}(t) from Equation 1 is determined for each sensor and is used to adapt the parameter settings of the AID system/MDA. At step 450, the processor may generate an adaptation parameter for use by a medication delivery algorithm or an AID system. For example, Equation 2 (reproduced below for convenience) may be used to determine the adaptation parameter that may be applied to the respective tunable AID system/MDA parameter settings. The adaptation parameter A(t) may be calculated according to Equation 2:

$$A ( t ) = \frac{{\sum }_{i = 1}^{n} {P}_{S n} ( t )}{n} \qquad ( Equation 2 )$$

where the adaptation parameter is an average of the available impact factors, where the summation is the sum of the respective values of the available impact factors PSn(t), and n is the number of sensors (i.e., number of impact factors).

The calculated adaptation parameter A(t) is then applied to each tunable parameter of the AID system/MDA as mentioned above. In a general summary, each sensor gets a weighted factor; then you have a weighted sum of each weighted factor (to combine all the sensors).

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, novel subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more features as variously disclosed or otherwise demonstrated herein.

What is claimed is:

1. A system, comprising:
a plurality of sensors, wherein each sensor is configured to generate a respective output related to a state of a user;
a memory storing a plurality of sensor baseline readings of respective sensors of the plurality of sensors; and
a processor operable to execute programming instructions, wherein the processor, when executing the programming instructions, is operable to:
obtain a respective sensor reading output from a respective sensor of the plurality of sensors;
evaluate the respective sensor reading and a respective sensor baseline reading that corresponds to the respective sensor in the stored plurality of sensor baseline readings; and
based on the evaluation, adjust an insulin delivery algorithm parameter.

2. The system of claim 1, wherein the processor, when evaluating the respective sensor reading and the respective sensor baseline reading that corresponds to the respective sensor in the stored plurality of sensor baseline readings, the processor is operable to:
determine a sensor reading difference between the respective sensor reading and the respective sensor baseline reading;
determine a maximum setting difference utilizing the respective sensor baseline reading;
calculate a preliminary parameter value using the determined sensor reading difference and the determined maximum setting difference; and
establish an insulin delivery parameter value for the respective sensor by applying a weighting to the calculated preliminary parameter value.

3. The system of claim 2, wherein the processor, when evaluating the respective sensor reading and a respective sensor baseline reading, is further operable to:
appraise a value of the insulin delivery algorithm parameter value that is to be adjusted; and
in response to the appraisal that the insulin delivery algorithm parameter is to be adjusted, produce a signal identifying the insulin delivery algorithm parameter value to be adjusted.

4. The system of claim 3, wherein the processor is further operable to:
in response to the signal, evaluate other respective sensor readings received from the other respective sensors of the plurality of sensors;
establish a respective insulin delivery parameter value for each of the other respective sensors of the plurality of sensors; and
using the insulin delivery parameter value and each respective insulin delivery parameter value of all of the other respective sensors of the plurality of sensors, generate an adaptation parameter usable by a medication delivery algorithm.

5. The system of claim 1, wherein the processor is further operable to:
calculate an insulin delivery parameter value for each of the plurality of sensors based on the respective sensor reading output received from each of the plurality of sensors;
use the insulin delivery parameter value for each of the plurality of sensors to calculate an adaptation parameter; and
recalculate a parameter setting of a medication delivery algorithm.

6. The system of claim 1, wherein the memory is further operable to store a medication delivery algorithm and the processor is further operable to execute the medication delivery algorithm.

7. The system of claim 1, further comprising:
a wearable drug delivery device including the memory, the processor, and communication circuitry, wherein the communication circuitry is operable to communicate wirelessly with one or more of the plurality of sensors.

8. The system of claim 1, further comprising:
a controller device including the memory, the processor, and communication circuitry, wherein the communication circuitry is operable to communicate wirelessly with one or more of the plurality of sensors.

9. The system of claim 1, wherein the plurality of sensors configured to detect a state of a user, comprise:
at least one of a wearable fitness monitor, a heart rate monitor, an oxygen sensor, a carbohydrate calculator, a pedometer, a meal tracker application, a blood glucose detector, or a ketone detector.

10. The system of claim 9, wherein one or more of the plurality of sensors further comprise:
communication circuitry operable to transmit a sensor reading indicative of the respective detected state.

11. The system of claim 1, wherein the sensor readings of a majority of the plurality of sensors are unused as a direct input to a drug delivery algorithm of the AID system or to a medication delivery algorithm.

12. A non-transitory computer-readable medium embodied with programming instructions that when executed by a processor, cause the processor to:
obtain a respective sensor reading output from a sensor;
evaluate the respective sensor reading and a respective sensor baseline reading that corresponds to the respective sensor of a plurality of sensor baseline readings; and
based on the evaluation, adjust an insulin delivery algorithm parameter.

13. The non-transitory computer-readable medium of claim 12, wherein the respective sensor reading output of the respective sensor of the plurality of sensors is unused as a direct input to a drug delivery algorithm of the AID system or to a medication delivery algorithm.

14. The non-transitory computer-readable medium of claim 12, wherein the processor, when evaluating the respective sensor reading and the respective sensor baseline reading that corresponds to the respective sensor in the stored plurality of sensor baseline readings, is further caused to:
determine a sensor reading difference between the respective sensor reading and the respective sensor baseline reading;
determine a maximum setting difference utilizing the respective sensor baseline reading;
calculate a preliminary parameter value using the determined sensor reading difference and the determined maximum setting difference; and
establish an insulin delivery parameter value for the respective sensor by applying a weighting to the calculated preliminary parameter value.

15. The non-transitory computer-readable medium of claim 12, wherein the processor, when evaluating the respective sensor reading and the respective sensor baseline reading that corresponds to the respective sensor in the stored plurality of sensor baseline readings, is further caused to:
evaluate a value of the insulin delivery algorithm parameter value that the generated indication that the insulin delivery algorithm parameter is to be adjusted; and
in response to the appraisal that the insulin delivery algorithm parameter is to be adjusted, produce a signal representing the generated indication that the insulin delivery algorithm parameter is to be adjusted.

16. The non-transitory computer-readable medium of claim 15, wherein the processor is further caused to:
in response to the signal, evaluate other respective sensor readings received from the other respective sensors of the plurality of sensors;
establish a respective insulin delivery parameter value for each of the other respective sensors of the plurality of sensors; and
using the insulin delivery parameter value and each respective insulin delivery parameter value of all of the other respective sensors of the plurality of sensors, generate an adaptation parameter usable by a medication delivery algorithm.

17. The non-transitory computer-readable medium of claim 16, wherein the processor is further caused to:
calculate an insulin delivery parameter value for each of the plurality of sensors based on the respective sensor reading output received from each of the plurality of sensors;
use the insulin delivery parameter value for each of the plurality of sensors to calculate an adaptation parameter; and
recalculate a parameter setting of a medication delivery algorithm.

18. A wearable drug delivery device, comprising:
a memory storing a plurality of sensor baseline readings of respective sensors of a plurality of sensors and a medication delivery algorithm;
communication circuitry operable to receive wireless signals; and
a processor coupled to the memory and the communication circuitry, the processor operable to execute programming instructions, wherein the processor, when executing the programming instructions, is operable to:
obtain sensor readings from one or more of the plurality of sensors;
based on the received sensor readings and corresponding baseline sensor readings, determine that the received sensor readings from one or more of the plurality of sensors indicate that a parameter input to a medication delivery algorithm is to be modified;
calculate an adaptation parameter based on a summation of an individual insulin delivery parameter generated for each respective one or more of the plurality of sensors; and
modify the parameter input to the medication delivery algorithm.

19. The wearable drug delivery device of claim 15, wherein processor, for each of the received sensor readings, is operable to:
determine a sensor reading difference between the respective sensor reading and the respective sensor baseline reading;
determine a maximum setting difference utilizing the respective sensor baseline reading;
calculate a preliminary parameter value using the determined sensor reading difference and the determined maximum setting difference; and
establish the insulin delivery parameter value for the respective sensor by applying a weighting to the calculated preliminary parameter value, wherein the insulin delivery parameter value for the respective sensor is included in the summation.

20. The wearable drug delivery device of claim 15, wherein communication circuitry is operable to:
receive signals from sensors external to a body of a user of the wearable device; and
provide the received signals to the processor, wherein the received signals represent sensor readings that are unused as direct inputs to a drug delivery algorithm executed by the processor.
